SEQUENCE LISTING (2 sequences)

SEQ ID NO: 1 - DNA, 20 bp, Artificial sequence
  Synthetic primer
  misc_feature (3)..(3): r = a or g
  misc_feature (7)..(7): y = t or c
  misc_feature (11)..(11): y = t or c
  misc_feature (12)..(12): m = a or c
agrgttygatymtggctcag

SEQ ID NO: 2 - DNA, 19 bp, Artificial sequence
  Synthetic primer
  misc_feature (3)..(3): y = t or c
ggytaccttgttacgactt

(19) United States
(12) Patent Application Publication
(10) Pub. No.: US 2022/0137056 A1
(43) Pub. Date: May 5, 2022

(54) ISOLATION AND DETECTION OF EXOSOME-ASSOCIATED MICROBIOME FOR DIAGNOSTIC AND THERAPEUTIC PURPOSES

(71) Applicant: Board of Regents, The University of Texas System, Austin, TX (US)

(72) Inventor: Raghu KALLURI, Houston, TX (US)

(73) Assignee: Board of Regents, The University of Texas System, Austin, TX (US)

(21) Appl. No.: 17/429,281

(22) PCT Filed: Feb. 7, 2020
(86) PCT No.: PCT/US20/17224

§ 371 Date: Aug. 6, 2021

Related U.S. Application Data
(60) Provisional application No. 62/802,994, filed on Feb. 8, 2019.

(51) Int. Cl. G01N 33/574; C12N 9/22

(52) U.S. Cl. CPC G01N 33/57488; G01N 2800/54; G01N 2800/52; C12N 9/22

(57) ABSTRACT

The present invention provides methods of predicting, diagnosing, and prognosing disease in a patient by analyzing the microbiome signature present in isolated exosomes. In one embodiment, provided herein are methods of detecting a microbiome in a patient, the method comprising: (a) obtaining a body fluid sample from a patient; (b) isolating an exosomes fraction of the body fluid sample; and (c) detecting a microbial macromolecule present in the exosomes fraction.

### REFERENCE TO RELATED APPLICATIONS

The present application claims the priority benefit of U.S. provisional application No. 62/802,994, filed Feb. 8, 2019, the entire contents of which is incorporated herein by reference.

### BACKGROUND

### 1. Field

The present invention relates generally to the field of medicine. More particularly, it concerns the detection of microbiome in circulating exosomes. Even more particularly, it concerns the detection of microbiome in circulating exosomes in the analysis and treatment of disease.

### 2. Description of Related Art

In recent years, microbiome present in human colon and other tissue has been identified as an important determinant for the health of an individual. In fact, tumor associated microbiome and colon associated microbiome has been identified as having an impact on cancer therapies, including immunotherapy. Methods for determining whether microbiome can impact the health of an individual and determine future risk for disease are needed.

### SUMMARY

Exosomes in the blood carry microbiome-related markers, such as nucleic acids. Therefore, the present invention provides methods of analyzing and detecting microbiome found in exosomes isolated from human serum samples.

In one embodiment, provided herein are methods of detecting a microbiome in a patient, the method comprising: (a) obtaining a body fluid sample from a patient; (b) isolating an exosomes fraction of the body fluid sample; and (c) detecting a microbial macromolecule present in the exosomes fraction. In some aspects, the body fluid sample is blood, lymph, saliva, sputum, urine, cerebrospinal fluid, bone marrow aspirates, eye exudate/tears, or serum.

In some aspects, the microbiome is a microbiome signature. In some aspects, the microbiome comprises two or more bacterial species. In some aspects, microbial macromolecule is a microbial nucleic acid molecule, such as, for example a microbial DNA molecule, a microbial 16S rRNA gene, or a microbial RNA molecule. In some aspects, the microbial macromolecule is a microbial protein.

In some aspects, the microbial signature indicates a risk factor for a disease. In some aspects, the microbial signature is compared to a microbial signature known to be associate with a disease. In some aspects, the microbial signature indicates a disease in the patient. In some aspects, the disease is a cancer, a genetic imprinting disorder, a neurological disorder, an autoimmune disease, or a metabolic disorder.

In some aspects, the disease is cancer and the methods further comprise isolating glypican 1-containing exosomes from the exosomes fraction. In some aspects, the cancer is a breast cancer, lung cancer, head & neck cancer, prostate cancer, esophageal cancer, tracheal cancer, brain cancer, liver cancer, bladder cancer, stomach cancer, pancreatic cancer, ovarian cancer, uterine cancer, cervical cancer, testicular cancer, colon cancer, rectal cancer or skin cancer.

In some aspects, the methods further comprise administering to the patient a therapeutic agent. In some aspects, the disease is cancer and the therapeutic agent is an anti-cancer therapy.

In some aspects, the methods further comprise reporting the diagnosis of the patient. In some aspects, reporting comprises preparing a written or electronic report. In some aspects, the methods further comprise providing the report to the patient, a doctor, a hospital, or an insurance company.

In some aspects, the patient is a healthy patient. In some aspects, the patient is in remission and the method is a method of detecting relapse. In some aspects, the patient is a human. In some aspects, the methods further comprise performing the method a second time. In some aspects, the second time is at least one day, one week, or one month after the initial performance of the method.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, the variation that exists among the study subjects, or a value that is within 10% of a stated value.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-B. Identification of microbial DNA in healthy serum-derived exosomes. Serum-derived exosome samples were treated with DNAse prior to DNA extraction to remove any freely circulating nucleic acids. DNA was isolated from the DNAse-treated exosomes derived from healthy serum (1 mL). The isolated DNA was PCR amplified with universal primers for the bacterial 16S ribosomal RNA gene (27F-B: AGRGTTYGATYMTGGCTCAG (SEQ ID NO: 1), 1492R: GGYTACCTTGTTACGACTT (SEQ ID NO: 2); ˜1500 bp for 16S rRNA gene). DNA from E. coli was used as a positive control. DNA from human cell lines Panc-1 and Fibroblasts BJ, as well as blank (no template DNA), were used as negative controls. The amplified DNA was analyzed by gel electrophoresis. FIG. 1A shows data from one repeat; FIG. 1B shows data for another repeat.

### DETAILED DESCRIPTION

Exosomes in the blood of healthy individuals contain bacterial microbiome. These exosomes can be generated by microbes in the body or by cells infected with bacteria. As such, a patient's microbiome can be assessed by isolation circulating exosomes and detecting the microbial components, such as microbial nucleic acids, present therein. This allows for a patient's microbiome to be sampled using simple blood exosome testing. The results of such testing can determine therapy options and fecal implant outcomes. The microbiome is more stable in the exosomes because it is protected from the cells of the immune system and also evades immune clearance. A patient's microbiome can represent the overall health status of the individual and offer potential insights into risk for many diseases. As such, patients can be screened for various diseases, such as cancer, or for response to therapy by sampling the patient's exo-microbiome.

### I. EXOSOMES

The terms “microvesicle” and “exosomes,” as used herein, refer to a membranous particle having a diameter (or largest dimension where the particles is not spheroid) of between about 10 nm to about 5000 nm, more typically between 30 nm and 1000 nm, and most typically between about 50 nm and 750 nm, wherein at least part of the membrane of the exosomes is directly obtained from a cell. Most commonly, exosomes will have a size (average diameter) that is up to 5% of the size of the donor cell. Therefore, especially contemplated exosomes include those that are shed from a cell.

Exosomes may be detected in or isolated from any suitable sample type, such as, for example, body fluids. As used herein, the term “isolated” refers to separation out of its natural environment and is meant to include at least partial purification and may include substantial purification. As used herein, the term “sample” refers to any sample suitable for the methods provided by the present invention. The sample may be any sample that includes exosomes suitable for detection or isolation. Sources of samples include blood, bone marrow, pleural fluid, peritoneal fluid, cerebrospinal fluid, urine, saliva, amniotic fluid, malignant ascites, broncho-alveolar lavage fluid, synovial fluid, breast milk, sweat, tears, joint fluid, and bronchial washes. In one aspect, the sample is a blood sample, including, for example, whole blood or any fraction or component thereof. A blood sample suitable for use with the present invention may be extracted from any source known that includes blood cells or components thereof, such as venous, arterial, peripheral, tissue, cord, and the like. For example, a sample may be obtained and processed using well-known and routine clinical methods (e.g., procedures for drawing and processing whole blood). In one aspect, an exemplary sample may be peripheral blood drawn from a subject with cancer.

Exosomes may also be isolated from tissue samples, such as surgical samples, biopsy samples, tissues, feces, and cultured cells. When isolating exosomes from tissue sources it may be necessary to homogenize the tissue in order to obtain a single cell suspension followed by lysis of the cells to release the exosomes. When isolating exosomes from tissue samples it is important to select homogenization and lysis procedures that do not result in disruption of the exosomes. Exosomes contemplated herein are preferably isolated from body fluid in a physiologically acceptable solution, for example, buffered saline, growth medium, various aqueous medium, etc.

Exosomes may be isolated from freshly collected samples or from samples that have been stored frozen or refrigerated. In some embodiments, exosomes may be isolated from cell culture medium. Although not necessary, higher purity exosomes may be obtained if fluid samples are clarified before precipitation with a volume-excluding polymer, to remove any debris from the sample. Methods of clarification include centrifugation, ultracentrifugation, filtration, or ultrafiltration. Most typically, exosomes can be isolated by numerous methods well-known in the art. One preferred method is differential centrifugation from body fluids or cell culture supernatants. Exemplary methods for isolation of exosomes are described in (Losche et al., 2004; Mesri and Altieri, 1998; Morel et al., 2004). Alternatively, exosomes may also be isolated via flow cytometry as described in (Combes et al., 1997).

One accepted protocol for isolation of exosomes includes ultracentrifugation, often in combination with sucrose density gradients or sucrose cushions to float the relatively low-density exosomes. Isolation of exosomes by sequential differential centrifugations is complicated by the possibility of overlapping size distributions with other microvesicles or macromolecular complexes. Furthermore, centrifugation may provide insufficient means to separate vesicles based on their sizes. However, sequential centrifugations, when combined with sucrose gradient ultracentrifugation, can provide high enrichment of exosomes.

Isolation of exosomes based on size, using alternatives to the ultracentrifugation routes, is another option. Successful purification of exosomes using ultrafiltration procedures that are less time consuming than ultracentrifugation, and do not require use of special equipment have been reported. Similarly, a commercial kit is available (EXOMIR™, Bioo Scientific) which allows removal of cells, platelets, and cellular debris on one microfilter and capturing of vesicles bigger than 30 nm on a second microfilter using positive pressure to drive the fluid. However, for this process, the exosomes are not recovered, their RNA content is directly extracted from the material caught on the second microfilter, which can then be used for PCR analysis. HPLC-based protocols could potentially allow one to obtain highly pure exosomes, though these processes require dedicated equipment and are difficult to scale up. A significant problem is that both blood and cell culture media contain large numbers of nanoparticles (some non-vesicular) in the same size range as exosomes. For example, some miRNAs may be contained within extracellular protein complexes rather than exosomes; however, treatment with protease (e.g., proteinase K) can be performed to eliminate any possible contamination with “extraexosomal” protein.

In another embodiment, cancer cell-derived exosomes may be captured by techniques commonly used to enrich a sample for exosomes, such as those involving immunospecific interactions (e.g., immunomagnetic capture). Immunomagnetic capture, also known as immunomagnetic cell separation, typically involves attaching antibodies directed to proteins found on a particular cell type to small paramagnetic beads. When the antibody-coated beads are mixed with a sample, such as blood, they attach to and surround the particular cell. The sample is then placed in a strong magnetic field, causing the beads to pellet to one side. After removing the blood, captured cells are retained with the beads. Many variations of this general method are well-known in the art and suitable for use to isolate exosomes. In one example, the exosomes may be attached to magnetic beads (e.g., aldehyde/sulphate beads) and then an antibody is added to the mixture to recognize an epitope on the surface of the exosomes that are attached to the beads. Exemplary proteins that are known to be found on cancer cell-derived exosomes include ATP-binding cassette sub-family A member 6 (ABCA6), tetraspanin-4 (TSPAN4), SLIT and NTRK-like protein 4 (SLITRK4), putative protocadherin beta-18 (PCDHB18), myeloid cell surface antigen CD33 (CD33), and glypican-1 (GPC1) (U.S. Pat. No. 9,921,223, which is incorporated herein by reference in its entirety). Cancer cell-derived exosomes may be isolated using, for example, antibodies or aptamers to one or more of these proteins.

As used herein, analysis includes any method that allows direct or indirect visualization of exosomes and may be in vivo or ex vivo. For example, analysis may include, but not limited to, ex vivo microscopic or cytometric detection and visualization of exosomes bound to a solid substrate, flow cytometry, fluorescent imaging, and the like. In an exemplary aspect, cancer cell-derived exosomes are detected using antibodies directed to one or more of ATP-binding cassette sub-family A member 6 (ABCA6), tetraspanin-4 (TSPAN4), SLIT and NTRK-like protein 4 (SLITRK4), putative protocadherin beta-18 (PCDHB18), myeloid cell surface antigen CD33 (CD33), glypican-1 (GPC1), Histone H2A type 2-A (HIST1H2AA), Histone H2A type 1-A (HIST1H1AA), Histone H3.3 (H3F3A), Histone H3.1 (HIST1H3A), Zinc finger protein 37 homolog (ZFP37), Laminin subunit beta-1 (LAMB1), Tubulointerstitial nephritis antigen-like (TINAGL1), Peroxiredeoxin-4 (PRDX4), Collagen alpha-2(IV) chain (COL4A2), Putative protein C3P1 (C3P1), Hemicentin-1 (HMCN1), Putative rhophilin-2-like protein (RHPN2P1), Ankyrin repeat domain-containing protein 62 (ANKRD62), Tripartite motif-containing protein 42 (TRIM42), Junction plakoglobin (JUP), Tubulin beta-2B chain (TUBB2B), Endoribonuclease Dicer (DICER1), E3 ubiquitin-protein ligase TRIM71 (TRIM71), Katanin p60 ATPase-containing subunit A-like 2 (KATNAL2), Protein S100-A6 (S100A6), 5′-nucleotidase domain-containing protein 3 (NT5DC3), Valine-tRNA ligase (VARS), Kazrin (KAZN), ELAV-like protein 4 (ELAVL4), RING finger protein 166 (RNF166), FERM and PDZ domain-containing protein 1 (FRMPD1), 78 kDa glucose-regulated protein (HSPA5), Trafficking protein particle complex subunit 6A (TRAPPC6A), Squalene monooxygenase (SQLE), Tumor susceptibility gene 101 protein (TSG101), Vacuolar protein sorting 28 homolog (VPS28), Prostaglandin F2 receptor negative regulator (PTGFRN), Isobutyryl-CoA dehydrogenase, mitochondrial (ACAD8), 26S protease regulatory subunit 6B (PSMC4), Elongation factor 1-gamma (EEF1G), Titin (TTN), Tyrosine-protein phosphatase type 13 (PTPN13), Triosephosphate isomerase (TPI1), or Carboxypeptidase E (CPE) and subsequently bound to a solid substrate and/or visualized using microscopic or cytometric detection.

It should be noted that not all proteins expressed in a cell are found in exosomes secreted by that cell. For example, calnexin, GM130, and LAMP-2 are all proteins expressed in MCF-7 cells but not found in exosomes secreted by MCF-7 cells (Baietti et al., 2012). As another example, one study found that 190/190 pancreatic ductal adenocarcinoma patients had higher levels of GPC1+ exosomes than healthy controls (Melo et al., 2015, which is incorporated herein by reference in its entirety). Notably, only 2.3% of healthy controls, on average, had GPC1+ exosomes.

A. Exemplary Protocol for Collecting Exosomes from Cell Culture

On Day 1, seed enough cells (e.g., about five million cells) in T225 flasks in media containing 10% FBS so that the next day the cells will be about 70% confluent. On Day 2, aspirate the media on the cells, wash the cells twice with PBS, and then add 25-30 mL base media (i.e., no PenStrep or FBS) to the cells. Incubate the cells for 24-48 hours. A 48 hour incubation is preferred, but some cells lines are more sensitive to serum-free media and so the incubation time should be reduced to 24 hours. Note that FBS contains exosomes that will heavily skew NanoSight results.

On Day 3/4, collect the media and centrifuge at room temperature for five minutes at 800×g to pellet dead cells and large debris. Transfer the supernatant to new conical tubes and centrifuge the media again for 10 minutes at 2000×g to remove other large debris and large vesicles. Pass the media through a 0.2 μm filter and then aliquot into ultracentrifuge tubes (e.g., 25×89 mm Beckman Ultra-Clear) using 35 mL per tube. If the volume of media per tube is less than 35 mL, fill the remainder of the tube with PBS to reach 35 mL. Ultracentrifuge the media for 2-4 hours at 28,000 rpm at 4° C. using a SW 32 Ti rotor (k-factor 266.7, RCF max 133,907). Carefully aspirate the supernatant until there is roughly 1-inch of liquid remaining. Tilt the tube and allow remaining media to slowly enter aspirator pipette. If desired, the exosomes pellet can be resuspended in PBS and the ultracentrifugation at 28,000 rpm repeated for 1-2 hours to further purify the population of exosomes.

Finally, resuspend the exosomes pellet in 210 μL PBS. If there are multiple ultracentrifuge tubes for each sample, use the same 210 μL PBS to serially resuspend each exosomes pellet. For each sample, take 10 μL and add to 990 μL H₂O to use for nanoparticle tracking analysis. Use the remaining 200 μL exosomes-containing suspension for downstream processes or immediately store at −80° C.

B. Exemplary Protocol for Extracting Exosomes from Serum Samples

First, allow serum (or other body fluid) samples to thaw on ice. Then, dilute 250 μL of cell-free serum samples in 11 mL PBS; filter through a 0.2 μm pore filter. Ultracentrifuge the diluted sample at 150,000×g overnight at 4° C. The following day, carefully discard the supernatant and wash the exosomes pellet in 11 mL PBS. Perform a second round of ultracentrifugation at 150,000×g at 4° C. for 2 hours. Finally, carefully discard the supernatant and resuspend the exosomes pellet in 100 μL PBS for analysis.

### II. MICROBIOME

The human microbiota consists of trillions of microorganisms including 150-200 prevalent and 1000 less common bacterial species, harboring over 100-fold more genes than those present in the human genome. The microbiota is composed predominantly of bacteria, yet also contains archaea, protozoa, and viruses. The microbiota performs vital functions essential to health maintenance, including food processing, digestion of complex indigestible polysaccharides and synthesis of vitamins, and it secretes bioactive metabolites with diverse functions, ranging from inhibition of pathogens, metabolism of toxic compounds to modulation of host metabolism.

A perturbed microbiota has been implicated in various disorders in humans, from necrotizing enterocolitis in infants, to obesity, diabetes, metabolic syndrome, irritable bowel syndrome, and inflammatory bowel disease in adults. Recent studies of microbiome dysbiosis in human health suggest specific changes in the microbiome in a number of disease states, including cancer. “Microbiome” refers to the collective genomes of a microbiota. Further, studies have suggested the association of a particular microbiome with specific cancers. Thus, a distinct microbiome may contribute to the cause or development of disease. Conversely, the tumor micro-environment may provide a specialized niche in which these viruses and microorganisms may persist. In either case, disease-type specific microbiome signatures may provide biomarkers for early diagnosis, prognosis, and treatment strategies.

In some embodiments, determining a level or set of levels of one or more types of microbes or components or products thereof comprises determining a level or set of levels of one or more DNA sequences. In some embodiments, one or more DNA sequences comprises any DNA sequence that can be used to differentiate between different microbial types. In certain embodiments, one or more DNA sequences comprises 16S rRNA gene sequences. In certain embodiments, one or more DNA sequences comprises 18S rRNA gene sequences. In some embodiments, 1, 2, 3, 4, 5, 10, 15, 20, 25, 50, 100, 1,000, 5,000 or more sequences are amplified.

16S and 18S rRNA gene sequences encode small subunit components of prokaryotic and eukaryotic ribosomes respectively. rRNA genes are particularly useful in distinguishing between types of microbes because, although sequences of these genes differ between microbial species, the genes have highly conserved regions for primer binding. This specificity between conserved primer binding regions allows the rRNA genes of many different types of microbes to be amplified with a single set of primers and then to be distinguished by amplified sequences.

### III. DIAGNOSIS, PROGNOSIS, AND TREATMENT OF DISEASES

Detection, isolation, and characterization of exo-microbiome, using the methods of the invention, is useful in assessing disease risk factors, diagnosis, and prognosis and in monitoring therapeutic efficacy for early detection of treatment failure that may lead to disease relapse. In addition, exo-microbiome analysis according to the invention enables the detection of early relapse in presymptomatic patients who have completed a course of therapy. This is possible because the presence of the microbiome present in exosomes may be associated and/or correlated with disease progression, poor response to therapy, relapse of disease, and/or decreased survival over a period of time. Thus, enumeration and characterization of exo-microbiome provides methods to stratify patients for baseline characteristics that predict initial risk and subsequent risk based upon response to therapy.

For example, cancer cell-derived exosomes isolated according to the methods disclosed above may be analyzed to diagnose or prognose cancer in the subject. As such, the methods of the present invention may be used, for example, to evaluate cancer patients and those at risk for cancer by comparing the exo-microbiome of cancer cell-derived exosomes and exosomes originated from non-cancerous cells. In any of the methods of diagnosis or prognosis described herein, either the presence or the absence of one or more indicators of cancer, such as a cancer-specific exo-microbiome signature, or of any other disorder, may be used to generate a diagnosis or prognosis.

In one aspect, a body fluid (e.g., blood, urine, saliva, etc.) sample is drawn from the patient and disease cell-derived exosomes are detected and/or isolated as described herein. For example, the exosomes may be labeled with one or more antibodies or aptamers that bind to ATP-binding cassette sub-family A member 6 (ABCA6), tetraspanin-4 (TSPAN4), SLIT and NTRK-like protein 4 (SLITRK4), putative protocadherin beta-18 (PCDHB18), myeloid cell surface antigen CD33 (CD33), and/or glypican-1 (GPC1), and the antibodies may have a covalently bound fluorescent label. Analysis may then be performed to determine the number and characterization of cancer cell-derived exosomes in the sample, and from this measurement, the number of cancer cell-derived exosomes present in the initial blood sample may be determined. Exosomes identified as cancer cell-derived exosomes may be verified as such through the detection of a second (or more) marker known to be found selectively or specifically in cancer cell-derived exosomes, such as, for example, Histone H2A type 2-A (HIST1H2AA), Histone H2A type 1-A (HIST1H1AA), Histone H3.3 (H3F3A), Histone H3.1 (HIST1H3A), Zinc finger protein 37 homolog (ZFP37), Laminin subunit beta-1 (LAMB1), Tubulointerstitial nephritis antigen-like (TINAGL1), Peroxiredeoxin-4 (PRDX4), Collagen alpha-2(IV) chain (COL4A2), Putative protein C3P1 (C3P1), Hemicentin-1 (HMCN1), Putative rhophilin-2-like protein (RHPN2P1), Ankyrin repeat domain-containing protein 62 (ANKRD62), Tripartite motif-containing protein 42 (TRIM42), Junction plakoglobin (JUP), Tubulin beta-2B chain (TUBB2B), Endoribonuclease Dicer (DICER1), E3 ubiquitin-protein ligase TRIM71 (TRIM71), Katanin p60 ATPase-containing subunit A-like 2 (KATNAL2), Protein S100-A6 (S100A6), 5′-nucleotidase domain-containing protein 3 (NT5DC3), Valine-tRNA ligase (VARS), Kazrin (KAZN), ELAV-like protein 4 (ELAVL4), RING finger protein 166 (RNF166), FERM and PDZ domain-containing protein 1 (FRMPD1), 78 kDa glucose-regulated protein (HSPA5), Trafficking protein particle complex subunit 6A (TRAPPC6A), Squalene monooxygenase (SQLE), Tumor susceptibility gene 101 protein (TSG101), Vacuolar protein sorting 28 homolog (VPS28), Prostaglandin F2 receptor negative regulator (PTGFRN), Isobutyryl-CoA dehydrogenase, mitochondrial (ACAD8), 26S protease regulatory subunit 6B (PSMC4), Elongation factor 1-gamma (EEF1G), Titin (TTN), Tyrosine-protein phosphatase type 13 (PTPN13), Triosephosphate isomerase (TPI1), or Carboxypeptidase E (CPE). The number of cancer cell-derived exosomes may be determined by cytometric or microscopic techniques to visually quantify and characterize the exosomes. Cancer cell-derived exosomes may be detected and quantified by other methods known in the art (e.g., ELISA).

In various aspects, analysis of a subject's exo-microbiome may be made over a particular time course in various intervals to assess a subject's progression and pathology. For example, analysis may be performed at regular intervals such as one day, two days, three days, one week, two weeks, one month, two months, three months, six months, or one year, in order to track the level and characterization of exo-microbiome as a function of time. In the case of existing cancer patients, this provides a useful indication of the progression of the disease and assists medical practitioners in making appropriate therapeutic choices based on the increase, decrease, or lack of change in exo-microbiome.

In any of the methods provided herein, additional analysis may also be performed to characterize exo-microbiome to provide additional clinical assessment. For example, PCR techniques may be employed, such as multiplexing with primers specific for particular markers to obtain information such as the type of microbe from which the exo-microbiome originated. Additionally, DNA or RNA analysis, proteome analysis, or metabolome analysis may be performed as a means of assessing additional information regarding characterization of the patient.

For example, an exo-microbiome analysis may provide data sufficient to make determinations of responsiveness of a subject to a particular therapeutic regime, or for determining the effectiveness of a candidate agent in the treatment of cancer. Accordingly, the present invention provides a method of determining responsiveness of a subject to a particular therapeutic regime or determining the effectiveness of a candidate agent in the treatment of cancer by detecting exo-microbiome of the subject as described herein. For example, once a drug treatment is administered to a patient, it is possible to determine the efficacy of the drug treatment using the methods of the invention. For example, a sample taken from the patient before the drug treatment, as well as one or more samples taken from the patient concurrently with or subsequent to the drug treatment, may be processed using the methods of the invention. By comparing the results of the analysis of each processed sample, one may determine the efficacy of the drug treatment or the responsiveness of the patient to the agent. In this manner, early identification may be made of failed compounds or early validation may be made of promising compounds.

Certain aspects of the present invention can be used to prevent or treat a disease or disorder based on the presence of exo-microbiome. Certain aspects of the present invention provide for treating a patient with exo-microbiome that express or comprise a therapeutic agent or a diagnostic agent. A “therapeutic agent” as used herein is an atom, molecule, or compound that is useful in the treatment of cancer or other conditions. Examples of therapeutic agents include, but are not limited to, drugs, chemotherapeutic agents, therapeutic antibodies and antibody fragments, toxins, radioisotopes, enzymes, nucleases, hormones, immunomodulators, antisense oligonucleotides, chelators, boron compounds, photoactive agents, and dyes. A “diagnostic agent” as used herein is an atom, molecule, or compound that is useful in diagnosing, detecting or visualizing a disease. According to the embodiments described herein, diagnostic agents may include, but are not limited to, radioactive substances (e.g., radioisotopes, radionuclides, radiolabels or radiotracers), dyes, contrast agents, fluorescent compounds or molecules, bioluminescent compounds or molecules, enzymes and enhancing agents (e.g., paramagnetic ions).

In some aspects, a therapeutic recombinant protein may be a protein having an activity that has been lost in a cell of the patient, a protein having a desired enzymatic activity, a protein having a desired inhibitory activity, etc. For example, the protein may be a transcription factor, an enzyme, a proteinaceous toxin, an antibody, a monoclonal antibody, etc. The monoclonal antibody may specifically or selectively bind to an intracellular antigen. The monoclonal antibody may inhibit the function of the intracellular antigen and/or disrupt a protein-protein interaction. Other aspects of the present invention provide for diagnosing a disease based on the presence of certain exo-microbiome found in cancer cell-derived exosomes in a patient sample.

The term “subject” as used herein refers to any individual or patient to which the subject methods are performed. Generally the subject is human, although as will be appreciated by those in the art, the subject may be an animal. Thus other animals, including mammals, such as rodents (including mice, rats, hamsters, and guinea pigs), cats, dogs, rabbits, farm animals (including cows, horses, goats, sheep, pigs, etc.), and primates (including monkeys, chimpanzees, orangutans, and gorillas) are included within the definition of subject.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of chemotherapy, immunotherapy, or radiotherapy, performance of surgery, or any combination thereof.

The term “therapeutic benefit” or “therapeutically effective” as used herein refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the invasiveness of a tumor, reduction in the growth rate of the cancer, or prevention of metastasis. Treatment of cancer may also refer to prolonging survival of a subject with cancer.

The term “cancer,” as used herein, may be used to describe a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus.

It is also recognized that the present invention may also be used to diagnose a non-cancerous disease, and in particular to diagnose any disease known to be associated with alterations in exo-microbiome. For example, the present invention may be used to diagnose an autoimmune disease (e.g., rheumatoid arthritis, systemic lupus erythematosus, multiple sclerosis), a metabolic disorder (hyperglycemia, hyperlipidemia, cardiovascular disease, diabetes), a neurological disease (e.g., autism spectrum disorder, Rett syndrome, Parkinson's disease, schizophrenia), or a psychological disorder.

An effective response of a patient or a patient's “responsiveness” to treatment refers to the clinical or therapeutic benefit imparted to a patient at risk for, or suffering from, a disease or disorder. Such benefit may include cellular or biological responses, a complete response, a partial response, a stable disease (without progression or relapse), or a response with a later relapse. For example, an effective response can be reduced tumor size or progression-free survival in a patient diagnosed with cancer.

Treatment outcomes can be predicted and monitored and/or patients benefiting from such treatments can be identified or selected via the methods described herein.

Regarding neoplastic condition treatment, depending on the stage of the neoplastic condition, neoplastic condition treatment involves one or a combination of the following therapies: surgery to remove the neoplastic tissue, radiation therapy, and chemotherapy. Other therapeutic regimens may be combined with the administration of the anticancer agents, e.g., therapeutic compositions and chemotherapeutic agents. For example, the patient to be treated with such anti-cancer agents may also receive radiation therapy and/or may undergo surgery.

For the treatment of disease, the appropriate dosage of a therapeutic composition will depend on the type of disease to be treated, as defined above, the severity and course of the disease, the patient's clinical history and response to the agent, and the discretion of the attending physician. The agent is suitably administered to the patient at one time or over a series of treatments.

Therapeutic and prophylactic methods and compositions can be provided in a combined amount effective to achieve the desired effect. A tissue, tumor, or cell can be contacted with one or more compositions or pharmacological formulation(s) comprising one or more of the agents, or by contacting the tissue, tumor, and/or cell with two or more distinct compositions or formulations. Also, it is contemplated that such a combination therapy can be used in conjunction with chemotherapy, radiotherapy, surgical therapy, or immunotherapy.

### IV. KITS AND DIAGNOSTICS

In various aspects of the invention, a kit is envisioned containing the necessary components to purify exosomes from a body fluid or tissue culture medium. In yet other aspects, a kit is envisioned containing the necessary components to isolate exosomes and determine the presence of microbiome within the isolated exosomes.

The kit may comprise one or more sealed vials containing any of such components. In some embodiments, the kit may also comprise a suitable container means, which is a container that will not react with components of the kit, such as an eppendorf tube, an assay plate, a syringe, a bottle, or a tube. The container may be made from sterilizable materials such as plastic or glass. The kit may further include an instruction sheet that outlines the procedural steps of the methods set forth herein, and will follow substantially the same procedures as described herein or are known to those of ordinary skill. The instruction information may be in a computer readable media containing machine-readable instructions that, when executed using a computer, cause the display of a real or virtual procedure of purifying exosomes from a sample, and/or identifying exo-microbiome therein.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Identification of Microbial DNA in Healthy Serum-Derived Exosomes

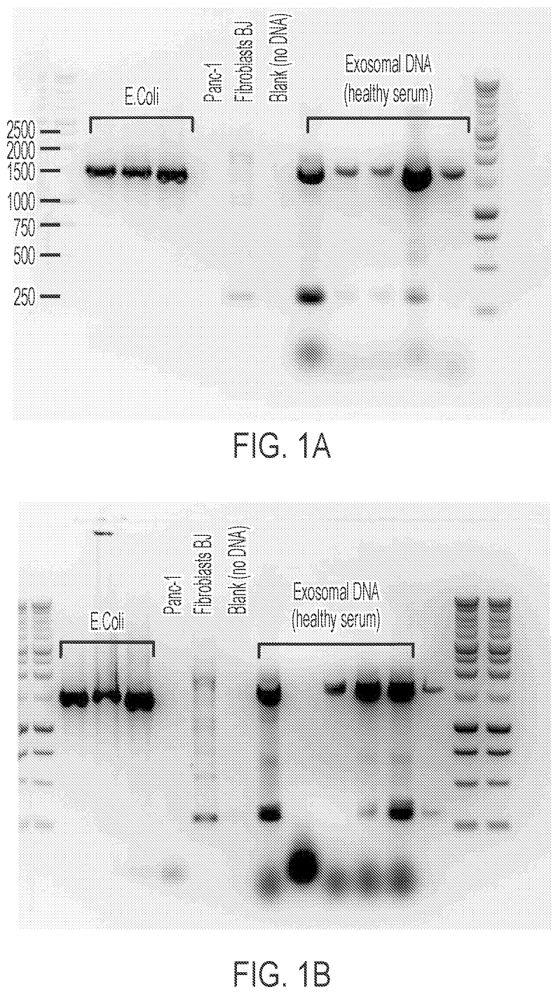

Exosomes were isolated from serum samples (1 mL) obtained from five healthy human subjects. The isolated exosomes were treated with DNAse prior to DNA extraction to remove any freely circulating nucleic acids in order to ensure intraluminal localization for any isolated DNA. Following DNA extraction, the DNA was PCR amplified with universal primers for the bacterial 16S ribosomal RNA gene (27F-B: AGRGTTYGATYMTGGCTCAG (SEQ ID NO: 1), 1492R: GGYTACCTTGTTACGACTT (SEQ ID NO: 2); ˜1500 bp for 16S rRNA gene). E. coli DNA was used as a positive control. DNA isolated from the human cell lines Panc-1 and Fibroblasts BJ, as well as a blank (no template DNA), were used as negative controls. The amplified DNA was visualized using gel electrophoresis. Data obtained from two repeats of this protocol are presented in FIGS. 1A-B.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 9,921,223
Baietti et al., Syndecan-syntenin-ALIX regulated the biogenesis of exosomes, Nat. Cell Biol., 14:677-685, 2012.
Combes et al., A new flow cytometry method of platelet-derived microvesicle quantitation in plasma, Thromb. Haemost., 77:220, 1997.
Losche et al., Platelet-derived microvesicles transfer tissue factor to monocytes but not to neutrophils, Platelets, 15: 109-115, 2004.
Melo et al., Glypican-1 identifies cancer exosomes and detects early pancreatic cancer, Nature, 523:177-182, 2015.
Mesri and Altieri, Endothelial cell activation by leukocyte microparticles, J. Immunol., 161:4382-4387, 1998.
Morel et al., Cellular microparticles: a disseminated storage pool of bioactive vascular effectors, Curr. Opin. Hematol., 11:156-164, 2004.

What is claimed is:

1. A method of detecting a microbiome in a patient, the method comprising:
(a) obtaining a body fluid sample from a patient;
(b) isolating an exosomes fraction of the body fluid sample; and
(c) detecting a microbial macromolecule present in the exosomes fraction.

2. The method of claim 1, wherein the microbiome is a microbiome signature.

3. The method of claim 1, wherein the microbiome comprises two or more bacterial species.

4. The method of claim 1, wherein the microbial macromolecule is a microbial nucleic acid molecule.

5. The method of claim 4, wherein the microbial nucleic acid molecule is a microbial DNA molecule.

6. The method of claim 5, wherein the microbial nucleic acid molecule is a microbial 16S rRNA gene.

7. The method of claim 4, wherein the microbial nucleic acid molecule is a microbial RNA molecule.

8. The method of claim 1, wherein the microbial macromolecule is a microbial protein.

9. The method of claim 2, wherein the microbial signature indicates a risk factor for a disease.

10. The method of claim 9, wherein the microbial signature is compared to a microbial signature known to be associate with a disease.

11. The method of claim 10, wherein the microbial signature indicates a disease in the patient.

12. The method of claim 1, wherein the patient is a healthy patient.

13. The method of claim 1, wherein the patient is in remission and the method is a method of detecting relapse.

14. The method of claim 1, wherein the body fluid sample is blood, lymph, saliva, sputum, urine, cerebrospinal fluid, bone marrow aspirates, eye exudate/tears, or serum.

15. The method of claim 11, wherein the disease is a cancer, a genetic imprinting disorder, a neurological disorder, an autoimmune disease, or a metabolic disorder.

16. The method of claim 11, wherein the disease is cancer, wherein the method further comprises isolating glypican 1-containing exosomes from the exosomes fraction.

17. The method of claim 16, wherein the cancer is a breast cancer, lung cancer, head & neck cancer, prostate cancer, esophageal cancer, tracheal cancer, brain cancer, liver cancer, bladder cancer, stomach cancer, pancreatic cancer, ovarian cancer, uterine cancer, cervical cancer, testicular cancer, colon cancer, rectal cancer or skin cancer.

18. The method of claim 11, further comprising administering to the patient a therapeutic agent.

19. The method of claim 18, wherein the disease is cancer, wherein the therapeutic agent is an anti-cancer therapy.

20. The method of claim 11, further comprising reporting the diagnosis of the patient.

21. The method of claim 20, wherein reporting comprises preparing a written or electronic report.

22. The method of claim 21, further comprising providing the report to the patient, a doctor, a hospital, or an insurance company.

23. The method of claim 1, wherein the patient is a human.

24. The method of claim 1, further comprising performing the method a second time.

25. The method of claim 24, wherein the second time is at least one day, one week, or one month after the initial performance of the method.
